(19) United States
(12) Patent Application Publication
(10) Pub. No.: US 2026/0263224 A1
(43) Pub. Date: Sep. 10, 2026

(54) INSERT FOR LOCKING SCREW ADAPTION

(71) Applicant: DEPUY IRELAND UNLIMITED COMPANY, Ringaskiddy Co.Cork (IE)

(72) Inventors: Philippa CLARKSON, Leeds (GB); David HORNE, Leeds (GB); Nazir-Ahmed KARBANEE, Leeds (GB); Karen MCKINLAY, Leeds (GB); Jonathan MEW, Leeds (GB); Charles WESTON, Leeds (GB); Enda LEAVY, Ringaskiddy Co.Cork (IE); Edward KAVANAGH, Ringaskiddy Co.Cork (IE); Joao Fernando ARRANHADO NEVES, Ringaskiddy Co.Cork (IE); James SEDICOL, Ringaskiddy Co.Cork (IE)

(21) Appl. No.: 19/166,563

(22) PCT Filed: Mar. 19, 2024
(86) PCT No.: PCT/EP2024/057355

§ 371 Date: Sep. 18, 2025

(30) Foreign Application Priority Data
Mar. 22, 2023 (GB) 2304153.6

(51) Int. Cl. A61F 2/34

(52) U.S. Cl. CPC A61F 2/34

(57) ABSTRACT

A surgical implant aperture insert for use in a surgical implant system; and a surgical implant system for use in joint arthroplasty is provided including an aperture and a surgical implant aperture insert. The insert increases the surface area of surgical components available for bone regrowth and can provide adaption means to allow the use of different fixings within the aperture.

### CROSS-REFERENCE

The present application is related to co-pending application serial number, presently unknown, filed on the same date as the present application at the same patent office by the same applicant, entitled “Insert for Setting Cement Mantle Thickness”. These applications and the patents arising therefrom are incorporated herein by reference, including their specification and drawings.

### FIELD

The present specification relates to an insert for positioning inside a screw hole of a surgical device. In particular, an insert for adapting a screw hole suitable for a non-locking into a screw hole suitable for a locking screw. The disclosure also relates to a kit including at least two implants of the disclosure. This disclosure also relates to a method of adapting a screw hole suitable for a non-locking screw into a screw hole suitable for a locking screw during surgery.

### BACKGROUND

Joint arthroplasty is a well-known surgical procedure by which a diseased and/or damaged natural joint is replaced by a prosthetic joint. For example, in a hip arthroplasty surgical procedure, a patient's natural hip joint is partially or totally replaced by a prosthetic hip prosthesis.

Occasionally revision hip arthroplasty surgery may need to be performed on a patient. In such a revision surgery, the previously implanted hip prosthesis is surgically removed, and a replacement hip prosthesis is implanted. In some revision surgeries, all of the components of the previously implanted hip prosthesis, including, for example, the femoral stem, neck component, and the acetabular cup, may be surgically removed. In other revision surgeries, only part of the previously implanted hip prosthesis may be removed and replaced.

The placement of locking and non-locking screws in surgical implants is determined depending on the type and location of the implant and whether the surgery is a revision surgery or not.

Typically, implants such as acetabular shells only provide screw holes suitable for a non-locking screw. This means that even if the surgeon would prefer to use a locking screw, it is not possible using the implant provided. This is because the dimensions of locking and non-locking screws are different. Locking screw holes are typically smaller and have tighter tolerances than non-locking screw holes.

Acetabular shells and other types of surgical implants used during revision surgeries typically contain a number of different screw holes, so that the surgeon has the option to choose a screw hole in a location with the most suitable bone. The surgeon will not typically place a screw in every available screw hole. The overall area of the surgical implant where bone ingrowth is facilitated is reduced by the number of screw holes present.

### SUMMARY

According to a first aspect of the disclosure there is provided a surgical implant system, the system comprising:
one or more surgical implants, at least one of the surgical implants having a distal surface and a proximal surface, a surgical implant aperture extending between the distal surface and the proximal surface;
one or more surgical fixings; and
one or more surgical implant aperture inserts;
wherein at least one of the surgical implant aperture inserts comprises:
a body portion, the body portion providing a distal surface and a proximal surface, the body portion further providing an intermediate surface extending between the distal surface and the proximal surface;
a through bore extending between the distal surface and the proximal surface, the through bore being defined by a through bore surface;
wherein the intermediate surface includes a surgical implant aperture contacting portion;
wherein the through bore surface includes a surgical fixing contacting portion.

The surgical implant aperture insert may further comprise, wherein the intermediate surface defines a first cross-sectional profile in a plane, wherein the through bore surface defines a second cross-sectional profile in that plane and wherein the second profile lies within first profile.

The surgical implant may be an orthopaedic implant. The surgical implant may be one or more of: an acetabular shell; a partial acetabular shell; a acetabular cage; a femoral implant. The surgical implant may be a trauma treating implant, for instance a trauma plate.

The surgical implant may be provided with an aperture surface provided with one or more insert retention elements.

The surgical implant may be provided with the one or more insert retention elements as male retention elements and/or female retention elements, for instance adapted to engage with complimentary elements provided on an insert, for instance on an intermediate surface of the insert. The surgical implant may be provided with a plurality of radially recessed female retention elements, such as grooves or recesses.

The surgical implant, for instance a surgical implant aperture thereof, may be provided with a threaded insert retention element.

The surgical kit may comprise two or more surgical fixings, including at least one fixing of a first type and at least one fixing of a second type. For instance, one or more screw fixings may be provided including one or more non-lockable fixings as the first type and/or one or more lockable fixings as the second type.

The first type of fixing may be different from the second type of fixing in terms of one or more characteristics of a threaded part of the fixing. One or more of the characteristics may be selected from: handedness; form; angle; lead; pitch; start; major diameter; minor diameter; pitch diameter; thread depth; or taper.

The first type of fixing may be different from the second type of fixing in terms of head profile and/or length and/or threaded section length and/or tip profile.

The first type of fixing may be different from the second type of fixing in terms of one or more dimensions of the fixing. One of the dimensions may include a diameter of the fixing, for instance of the part contacting the aperture in the insert. One of the dimensions may include the cross-sectional profile of the fixing, for instance the part contacting the aperture in the insert.

The surgical kit may comprise a plurality of different surgical implant aperture inserts. The inserts may be different from one another in terms of one or more or all of: the body portion; the distal surface; the proximal surface; the intermediate surface extending between the distal surface and the proximal surface; the through bore extending between the distal surface and the proximal surface; the through bore surface; the surgical implant aperture contacting portion; the surgical fixing contacting portion. The difference may be in terms of one or more or all of: a material composition; a porosity; a dimension; a separation between features; a size of the feature; an extent of a feature; a shape or profile of a feature.

The difference may be or include an inclination for the bore. The insert may have an insert axis. The insert axis may extend distally to proximally, for instance perpendicular to the proximal surface of the insert and/or perpendicular to the distal surface of the insert. The through bore may have a bore axis. The bore axis may be parallel to and for instance correspond to the insert axis. The bore axis may be inclined or have an inclination relative to the insert axis. The inclination may be between 0 degrees and 25 degrees relative to the insert axis. The inclination may be between 0 degrees and 17 degrees relative to the insert axis.

The first aspect of the disclosure may include any of the features, options and possibilities set out elsewhere in this document, including in the other aspects and description of the embodiments. In particular, the surgical implant aperture insert may include any of the features, options or possibilities set out in the second aspect.

According to a second aspect of this disclosure there is provided a surgical implant aperture insert, the insert comprising:
a body portion, the body portion providing a distal surface and a proximal surface, the body portion further providing an intermediate surface extending between the distal surface and the proximal surface;
a through bore extending between the distal surface and the proximal surface, the through bore being defined by a through bore surface;
wherein the intermediate surface includes a surgical implant aperture contacting portion;
wherein the through bore surface includes a surgical fixing contacting portion;
wherein the intermediate surface defines a first cross-sectional profile in a plane, wherein the through bore surface defines a second cross-sectional profile in that plane and wherein the second profile lies within first profile.

At least a part of the insert may be formed of a porous material. The porous material may be selected and/or be adapted to promote bone ingrowth.

At least a part of the insert may be formed of a deformable material. The deformable material may be selected and/or adapted to deform on engagement by a surgical fixing.

The deformable material may be selected and/or adapted to deform on engagement with a surgical implant aperture.

The porous material and the deformable material may be the same material or may be different materials.

The insert may provide a first part of the body portion that is formed of a first material and a second part of the body portion is formed of a second material. The first and second materials may be different from each other in their porosity and/or deformability. The first part of the body portion may define the through bore surface. The second part of the body portion may define the intermediate surface. The distal surface and/or the proximal surface may be partially defined by the first material and partially defined by the second material. The body portion may include a core of first material. The body portion may include a core surrounded radially by a second material.

At least a part of the insert may be formed of a deformable material, the deformable material having a first pre-deformation state for the through bore surface. The deformable material may have a first post-deformation state for the through bore surface. The shape of the through bore surface may be different in the first post-deformation state to the shape of the through bore surface in the first pre-deformation state. For instance, the shape of the through bore surface may be deformed to engage with a surgical fixing, for instance one or more contact surfaces thereof, for instance a screw thread provided by the surgical fixing, for instance a head portion of the surgical fixing. The insert may provide the through bore surface in the first post-deformation state is adapted to engage with one or more surfaces of a surgical fixing.

The insert may provide that the through bore surface includes a bone engaging surgical fixing contacting portion. The contacting portion may be provided by deformation of the through bore surface and/or by a thread provided by the through bore surface and/or by the surgical fixing cutting a thread in the through bore surface.

At least a part of the insert may be formed of a deformable material, the deformable material having a second pre-deformation state for the intermediate surface. The deformable material may have a second post-deformation state for the intermediate surface. The shape of the intermediate surface may be different in the second post-deformation state to the shape of the intermediate surface in the second pre-deformation state. For instance, the shape of the intermediate surface may be deformed to engage with a surgical implant aperture, for instance one or more contact surfaces thereof, for instance a through hole thereof. The insert may provide the through intermediate surface in the second post-deformation state is adapted to engage with one or more surfaces of a surgical implant aperture. The one or more surfaces may include or be: a surface defining the surgical implant aperture; a surface defining a through bore in the surgical implant; a distal surface of the surgical implant; a proximal surface of the surgical implant.

One or both of the distal surface and/or the proximal surface of the insert may shaped to be a continuation of and/or complimentary to a section of the surgical implant, for instance to the profile of an adjacent, in use, section of a surgical implant. The section of the surgical implant may be planar or may be curved, for instance part hemispherical. The curvature of one or both of the distal surface and/or the proximal surface of the insert may have a radii, for instance the same radii as a section of the surgical implant.

One or both of the distal surface and/or the proximal surface of the insert may shaped to be flush with a section of the surgical implant, for instance to the profile of an adjacent, in use, section of a surgical implant. The section of the surgical implant may be planar or may be curved, for instance part hemispherical.

One or both of the distal surface and/or the proximal surface of the insert may shaped to be recessed relative to a section of the surgical implant, for instance to the profile of an adjacent, in use, section of a surgical implant. The section of the surgical implant may be planar or may be curved, for instance part hemispherical. The curvature of one or both of the distal surface and/or the proximal surface of the insert may have a radii, for instance a radii which is greater that the radii of the section of the surgical implant.

One or both of the distal surface and/or the proximal surface of the insert may shaped to be proud of a section of the surgical implant, for instance proud of the profile of an adjacent, in use, section of a surgical implant. The proud element of the insert may be a head section or a flange section. The section of the surgical implant may be planar, for instance a flange, or may be curved, for instance a plate configured to correspond with a bone location.

The intermediate surface may define a first cross-sectional profile in a plane which is a regular shape, for instance a circle. The through bore surface may define a second cross-sectional profile in that plane which is a regular shape, potentially the same type of regular shape, for instance a circle. The regular shapes may be concentric, for instance concentric circles. The insert may provide that the first cross-sectional profile in a plane is a circular profile. The insert may provide that the second cross-sectional profile in that plane is also a circular profile. The insert may provide that the second cross-sectional profile has a diameter less than that of the first cross-sectional profile.

The insert may provide that the cross-section of the second cross-sectional profile reduces along the axis of the through bore and/or body portion axis. The second cross-sectional profile may taper away from the distal end and/or towards the proximal end, for instance by tapering of the bore from distal to proximal ends.

The insert may provide a tapered through bore, for instance with a screw thread.

The insert may provide that the cross-section of the first cross-sectional profile reduces along the axis of the through bore and/or along the axis of the body portion. The profile may reduce due to the tapering of the intermediate surface over part of the transition from distal to proximal ends. The intermediate surface may have a maximum first cross-sectional profile at the distal surface. The intermediate surface may have a minimum first cross-sectional profile at the proximal surface. The intermediate surface may have a minimum first cross-sectional profile from an intermediate position to the proximal surface, for instance to provide a cylindrical section. The intermediate surface may transition from the maximum cross-sectional profile to a minimum cross-sectional profile to provide a curved intermediate surface, for instance a bowl like section.

The insert may have an insert axis. The insert axis may extend distally to proximally, for instance perpendicular to the proximal surface of the insert and/or perpendicular to the distal surface of the insert. The through bore may have a bore axis. The bore axis may be parallel to and for instance correspond to the insert axis. The bore axis may be inclined or have an inclination relative to the insert axis.

A plurality of different orientations for the inclination may be provided, for instance relative to the insert and/or relative to the surgical implant, by the insert having a plurality of rotational positions. The insert may have a first rotational position for a first orientation of the inclination and a second rotational position for a second orientation of the inclination. The inclination for an insert may be the same in one or more or all of a plurality of rotational positions.

The inclination may be between 0 degrees and 25 degrees relative to the insert axis. The inclination may be between 0 degrees and 17 degrees relative to the insert axis.

The insert may be provided with an intermediate surface provided with one or more surgical implant retention elements.

The insert may be provided with the one or more surgical implant retention elements as male retention elements and/or female retention elements, for instance adapted to engage with complimentary elements provided on a surgical implant, for instance within a surface defining a surgical implant aperture. The insert may be provided with a plurality of radially extending male retention elements, such as cylindrical projections.

The insert may be provided with a threaded surgical implant retention element.

The insert may be a 3D printed insert. The insert may be 3D printed with one or more other components, for instance the surgical implant aperture and/or surgical implant.

The second aspect of the disclosure may include any of the features, options and possibilities set out elsewhere in this document, including in the other aspects and description of the embodiments.

According to a third aspect of the disclosure there is provided a method of configuring a surgical implant aperture in a surgical implant, the method comprising
a) selecting a surgical fixing to insert into a surgical implant aperture;
b) inserting a surgical implant aperture insert into the surgical implant aperture;
c) inserting the surgical fixing through the insert and through the aperture, wherein the insert comprises:
   a body portion, the body portion providing a distal surface and a proximal surface, the body portion further providing an intermediate surface extending between the distal surface and the proximal surface;
   a through bore extending between the distal surface and the proximal surface, the through bore being defined by a through bore surface;
   wherein the intermediate surface includes a surgical implant aperture contacting portion which contacts the surgical implant aperture after insertion of the insert into the aperture;
   wherein the through bore surface includes a surgical fixing contacting portion which contacts the surgical fixing after insertion of the surgical fixing into the bore.

The method may include that the insert further provides that the intermediate surface defines a first cross-sectional profile in a plane, wherein the through bore surface defines a second cross-sectional profile in that plane and wherein the second profile lies within first profile.

The method may include that the insert provides at least a part of the insert formed of a deformable material. The method may include a deformable material being deformed by engagement by the surgical fixing with the deformable material and/or by insertion of the surgical fixing into the through bore.

The method may further include that the insert provides at least a part of the insert formed of a deformable material, the deformable material having a first pre-deformation state for the through bore surface and a first post-deformation state for the through bore surface. The method may further provide that the shape of the through bore surface is different in the first post-deformation state to the shape of the through bore surface in the first pre-deformation state. The difference in shape may be caused by engagement by the surgical fixing with the deformable material and/or by insertion of the surgical fixing into the through bore.

The method may further include that the insert provides a through bore surface in the first post-deformation state which engages with one or more surfaces of a surgical fixing.

The method may provide that the through bore surface includes a bone engaging surgical fixing contacting portion.

The method may further provide that the insert provides at least a part of the insert formed of a deformable material, the deformable material having a second pre-deformation state for the intermediate surface and a second post-deformation state for the intermediate surface. The method may further provide that the shape of the intermediate surface is different in the second post-deformation state to the shape of the intermediate surface in the second pre-deformation state. The difference in shape may be caused by engagement by the surgical implant aperture with the deformable material and/or by insertion of the insert into the surgical implant aperture.

The method may further provide that insert provides an intermediate surface in the second post-deformation state which engages with one or more surfaces of a surgical implant aperture.

The method may include selecting:
a) one or more surgical implant apertures to receive a first size or first type of fixing, for instance for insertion into an insert in a surgical implant aperture;
b) one or more surgical implant apertures to receive an insert, but not receive a surgical fixing;
c) one or more surgical implant apertures to receive a second size or second type of fixing, for instance for insertion into a surgical implant aperture devoid of an insert.

The method may include providing different types of insert in two or more apertures. The different types may be different from one another in terms of one or more or all of: the body portion; the distal surface; the proximal surface; the intermediate surface extending between the distal surface and the proximal surface; the through bore extending between the distal surface and the proximal surface; the through bore surface; the surgical implant aperture contacting portion; the surgical fixing contacting portion. The difference may be, for such features, in terms of one or more or all of: a material composition; a porosity; a dimension; a separation between features; a size of the feature; an extent of a feature; a shape or profile of a feature.

The difference may be or include an inclination for the bore. The insert may have an insert axis. The insert axis may extend distally to proximally, for instance perpendicular to the proximal surface of the insert and/or perpendicular to the distal surface of the insert. The through bore may have a bore axis. The bore axis may be parallel to and for instance correspond to the insert axis. The bore axis may be inclined or have an inclination relative to the insert axis. The inclination may be between 0 degrees and 25 degrees relative to the insert axis. The inclination may be between 0 degrees and 17 degrees relative to the insert axis.

The difference may be a rotational position for the insert, particularly if provided with an inclined bore.

The third aspect of the disclosure may include any of the features, options and possibilities set out elsewhere in this document, including in the other aspects and description of the embodiments.

According to a fourth aspect of the disclosure there is provided a method of implanting a surgical implant, the method comprising:
a) selecting the surgical implant, the surgical implant having at least one surgical implant aperture;
b) implanting the surgical implant at a surgical site in a patient;
c) selecting a surgical fixing to insert into a surgical implant aperture;
d) selecting a surgical implant aperture insert to insert in the aperture of the surgical implant;
e) inserting the surgical implant aperture insert into the aperture of the surgical implant;
f) inserting the surgical fixing through the insert and through the aperture and into a site in the patient;
wherein the insert comprises:
   a body portion, the body portion providing a distal surface and a proximal surface, the body portion further providing an intermediate surface extending between the distal surface and the proximal surface;
   a through bore extending between the distal surface and the proximal surface, the through bore being defined by a through bore surface;
   wherein the intermediate surface includes a surgical implant aperture contacting portion which contacts the surgical implant aperture after insertion of the insert into the aperture;
   wherein the through bore surface includes a surgical fixing contacting portion which contacts the surgical fixing after insertion of the surgical fixing into the bore.

The method may include that the insert further provides that the intermediate surface defines a first cross-sectional profile in a plane, wherein the through bore surface defines a second cross-sectional profile in that plane and wherein the second profile lies within first profile.

The method may include that the insert provides at least a part of the insert formed of a deformable material. The method may include a deformable material being deformed by engagement by the surgical fixing with the deformable material and/or by insertion of the surgical fixing into the through bore.

The method may further include that the insert provides at least a part of the insert formed of a deformable material, the deformable material having a first pre-deformation state for the through bore surface and a first post-deformation state for the through bore surface. The method may further provide that the shape of the through bore surface is different in the first post-deformation state to the shape of the through bore surface in the first pre-deformation state. The difference in shape may be caused by engagement by the surgical fixing with the deformable material and/or by insertion of the surgical fixing into the through bore.

The method may further include that the insert provides a through bore surface in the first post-deformation state which engages with one or more surfaces of a surgical fixing.

The method may provide that the through bore surface includes a bone engaging surgical fixing contacting portion.

The method may further provide that the insert provides at least a part of the insert formed of a deformable material, the deformable material having a second pre-deformation state for the intermediate surface and a second post-deformation state for the intermediate surface. The method may further provide that the shape of the intermediate surface is different in the second post-deformation state to the shape of the intermediate surface in the second pre-deformation state. The difference in shape may be caused by engagement by the surgical implant aperture with the deformable material and/or by insertion of the insert into the surgical implant aperture.

The method may further provide that insert provides an intermediate surface in the second post-deformation state which engages with one or more surfaces of a surgical implant aperture.

The method may include selecting:
d) one or more surgical implant apertures to receive a first size or first type of fixing, for instance for insertion into an insert in a surgical implant aperture;
e) one or more surgical implant apertures to receive an insert, but not receive a surgical fixing;
f) one or more surgical implant apertures to receive a second size or second type of fixing, for instance for insertion into a surgical implant aperture devoid of an insert.

The method may include providing different types of insert in two or more apertures. The different types may be different from one another in terms of one or more or all of: the body portion; the distal surface; the proximal surface; the intermediate surface extending between the distal surface and the proximal surface; the through bore extending between the distal surface and the proximal surface; the through bore surface; the surgical implant aperture contacting portion; the surgical fixing contacting portion. The difference may be, for such features, in terms of one or more or all of: a material composition; a porosity; a dimension; a separation between features; a size of the feature; an extent of a feature; a shape or profile of a feature.

The difference may be or include an inclination for the bore. The insert may have an insert axis. The insert axis may extend distally to proximally, for instance perpendicular to the proximal surface of the insert and/or perpendicular to the distal surface of the insert. The through bore may have a bore axis. The bore axis may be parallel to and for instance correspond to the insert axis. The bore axis may be inclined or have an inclination relative to the insert axis. The inclination may be between 0 degrees and 25 degrees relative to the insert axis. The inclination may be between 0 degrees and 17 degrees relative to the insert axis.

The difference may be a rotational position for the insert, particularly if provided with an inclined bore.

The third aspect of the disclosure may include any of the features, options and possibilities set out elsewhere in this document, including in the other aspects and description of the embodiments.

### BRIEF DESCRIPTION OF THE DRAWINGS

Embodiments of this disclosure will be described hereinafter, by way of example only, with reference to the accompanying drawings in which like reference signs relate to like elements and in which:

FIG. 1 is an illustration of a variety of surgical implants for which the disclosure is useful;

FIG. 2a a side view of an interference-fit insert according to a first embodiment;

FIG. 2b is a cross-sectional view of the interference-fit insert of FIG. 2a;

FIG. 2c is the cross-sectional view of FIG. 2b with a fixing present;

FIG. 3 is an illustration of a variety of sizes and types of fixing which can be used with the disclosure

FIG. 4 is a cross-sectional elevation of a further embodiment of insert, shown inserted into a through hole of an acetabular shell;

FIG. 5a is an illustration of a further embodiment of the disclosure, used in the context of an acetabular cage;

FIG. 5b is an illustration of a further embodiment of the disclosure, also used in the context of an acetabular shell, with the insert incorporating a flange engaging head;

FIGS. 6a to 6e are illustrations of further embodiments of the disclosure in which different parts of the insert are formed of different materials and/or in different manners;

FIG. 7a is a side view of an insert with mating features;

FIG. 7b is a perspective view of an acetabular cup with a screw hole having mating features and an insert with mating features inserted into a screw hole of the acetabular shell;

FIG. 8a is a cross-sectional side view of an embodiment of the insert with a first through hole orientation; and

FIG. 8b is a cross-sectional side view of an embodiment of the insert with a second through hole orientation.

### DETAILED DESCRIPTION

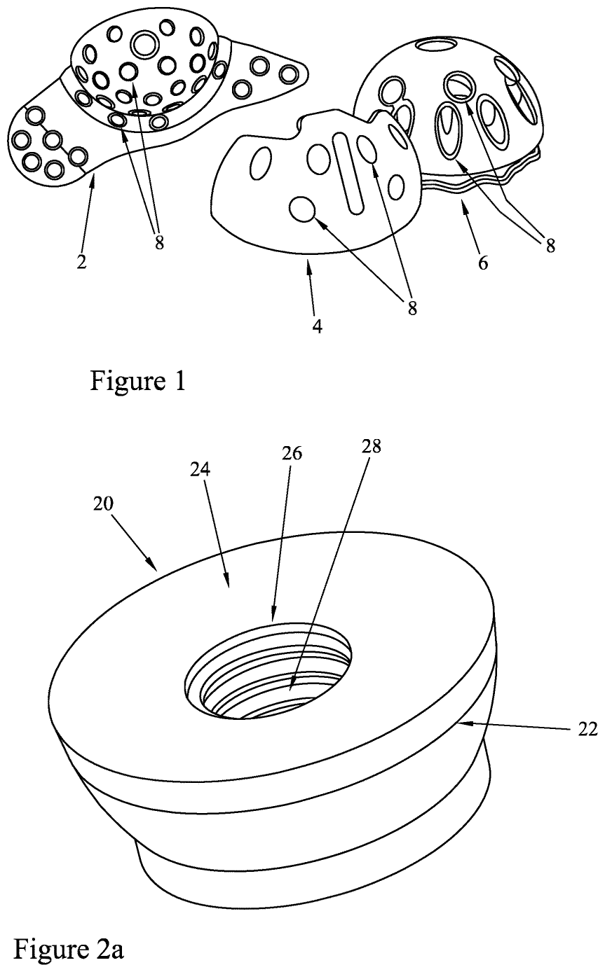

Referring to the drawings, FIG. 1 shows a series of perspective views of examples of surgical implants, these being an acetabular revision cage 2, partial acetabular cup 4 and acetabular cup 6. Each of these surgical implants 2, 4, 6 are provides with a significant number of through holes 8 to accommodate fixings [not shown] in use. Only a small number of the through holes 8 would be used in most surgical instances, with the others being left as open through holes 8.

These are just some examples of the wide variety of surgical implants that the disclosure is relevant to, as for example, the disclosure can be used in trauma implants, such as trauma plates, as well as in other orthopaedic examples, such as femoral implants, including distal locking femoral stems.

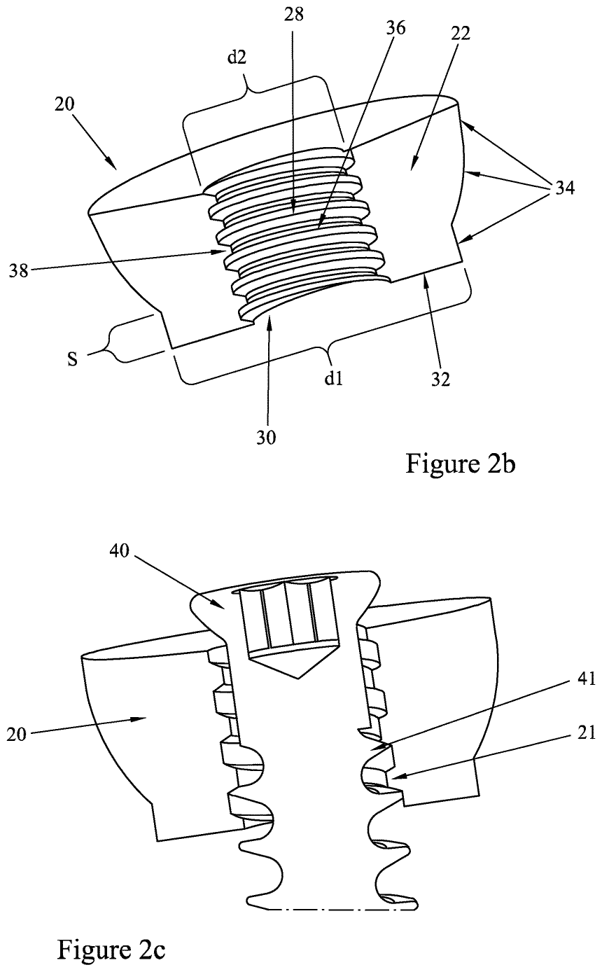

FIG. 2a provides a side perspective view of an embodiment of an insert 20 described with reference to an acetabular context of use. The insert 20 comprises a body portion 22 having a distal surface 24 which is provided with a distal opening 26 located at the distal end of a through bore 28 which extends through the body portion 22 to a proximal opening 30 provided in a proximal surface 32 of the body portion 22; see FIG. 2b.

The insert 20 is suitable for insertion into the through hole 8 of a variety of surgical implants, for example an acetabular shell, cage, a trauma plate, and/or a distal locking femoral stem.

To retain the insert 20 in the surgical implant 2, 6, 8, the outside diameter d1 of at least a section S of the insert 20 is an interference fit with a complimentary section of the surgical implant 2, 4, 6. The section could be another section to the section S illustrated or could be the whole of the profile of the outer surface 34. This retention provides a first level of retention of the insert 20 relative to the surgical implant 2, 6, 8. A further and increased level of retention is described below.

The insert 20 is formed from a porous material, for promoting bone ingrowth. In this embodiment, the entire body portion 22 is formed from a porous material, for promoting bone ingrowth. Thus, the provision of the insert 20 in a through hole 8 in the surgical component 2, 4, 6 has a benefit in increasing the surface area of the surgical component 2, 4, 6 available for bone ingrowth. The insert 20 can be used largely to fill the through hole 8, even if no further use of the insert 20 is made.

The insert 20 can also act as an adaptor to allow a different size and/or type of fixing to cooperate with the insert 20, and hence the surgical implant 2, 4, 6, than would normally cooperate with the surgical implant 2, 4, 6 directly by being inserted in a through hole 8 without any insert 20 present. The through bore 28 has a diameter d₂ which is less than a diameter of the through hole 8 of the surgical device 2, 4, 6; which is equivalent to the external diameter d₁ of the insert 20 plus a tolerance. Typical diameters for d₂ would be 2 mm to 3.5 mm, as examples only.

Whilst the through hole 8, without the insert 20 present, can securely receive fixings with an external diameter matching d₁, with the insert 20 present, the through bore 28 allows a smaller diameter fixing, such as a locking screw, to be engaged securely with the through bore 8 and hence with the surgical implant 2, 4, 6. This provides a second enhanced level of retention. This means that the surgeon can select those through holes 8 from amongst the full set of such through holes 8, which are: best to receive the larger size or first type of fixing; best to receive the smaller size or second type of fixing and so should have an insert 20 provided; and those through holes 8 which do not need to be provided with a fixing, but in which an insert 20 is to be provided to promote bone ingrowth. The surgeon may still select some through holes 8 not to receive a fixing or an insert 20. This flexibility of selection allows for good stability for the implant, optimal fixing sixes and/or types to be used and maximum bone ingrowth to be promoted.

FIG. 2b shows a thread 36 formed in the entire length of the side wall 38. In other examples which are not shown, the thread 36 may only extend part way along the length of the side wall 38. The thread 36 in FIGS. 2a and 2b has a constant diameter and is complementary to the external thread of a fixing, such as a locking screw.

As seen in FIG. 2c, when a fixing 40 is inserted into this form of insert 20, the threads 41 and 21 respectively engage one another and there is no deformation of the material forming either the insert 20 or the fixing 40.

The insert 20 in any of the embodiments may be 3D printed. The insert 20 may be 3D printed so that it is already in situ within the through hole 8 of a surgical implant 2, 4, 6, or can be inserted separately into a specific through hole 8.

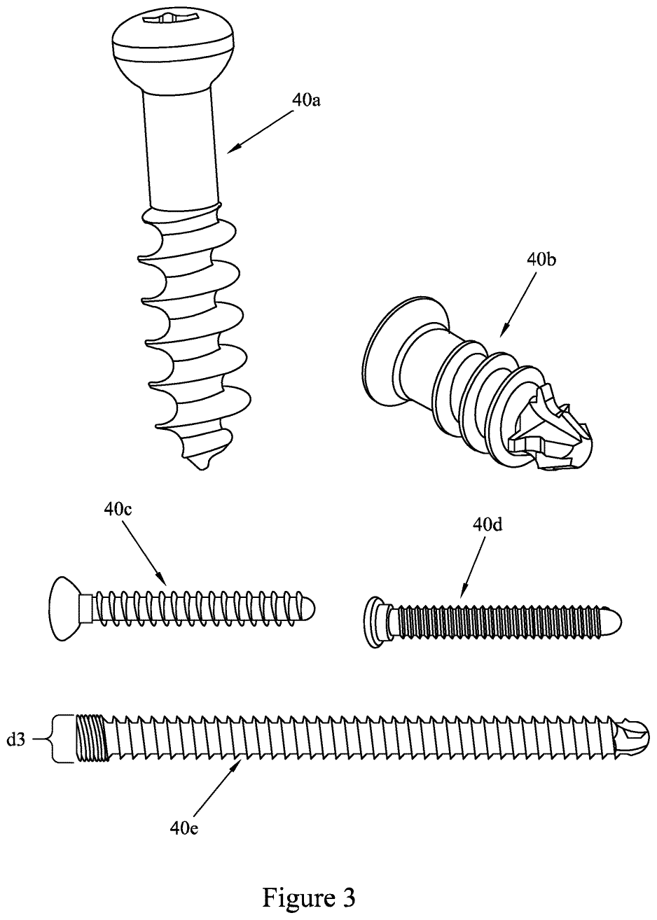

FIG. 3 illustrates a series of typical fixings 40a, 40b, 40c, 40d and 40e for cooperating directly with the surgical implant 2, 4, 6 which in a reduced diameter form can cooperate with the insert 20, as an adapter, and hence the surgical component 2, 4, 6.

The example fixing 40e is a locking screw. The diameter d₂ of the through bore 28 corresponds to the diameter d₃ of a locking screw head 30, but with the diameter d₃ increased slightly to cause deformation of the insert 20 as the fixing is inserted. The level of deformation of the surface of the insert 20 against the implant substrate is purely enough to gain an interference fit, for instance in the range of 1-5%. The insert 20 is slightly oversized to allow for the deformation. The deformation is controlled by the make-up of the insert 20 and by its structure. This can be via porosity, strut length and thickness, for instance, if the insert is 3D printed, or, if for instance if material is solid, it can be via the material properties.

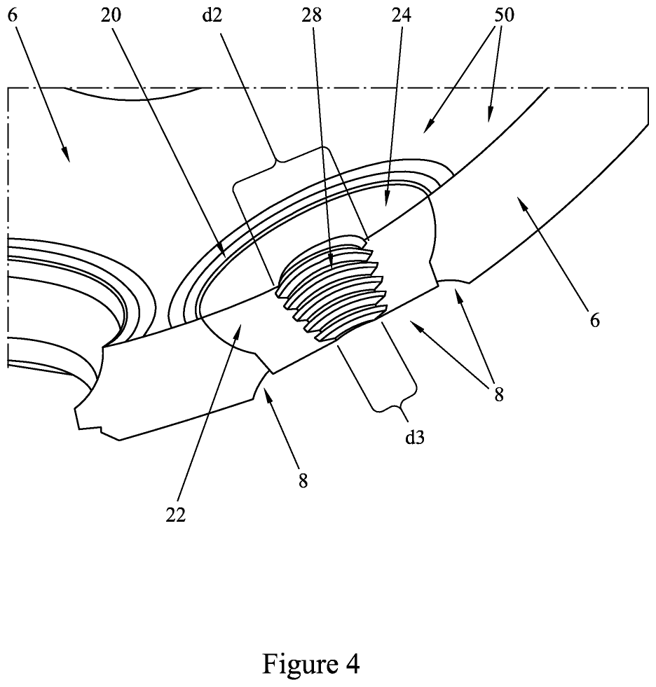

As shown in FIG. 4, the disclosure provides a further embodiment in which the insert 20 is shown in position within an opening, such as a through hole 8, in a surgical implant in the form of an acetabular cup 6 in this instance.

The geometry of the insert 20 is shaped so that its distal surface 24 is complimentary to and represents a continuation of the surrounding distal surface 50 of the acetabular cup 6 in which it is inserted. In this way, no interference with the intended profile of the distal surface 50 occurs and no interference with the opposing part of the surgical implant system, not shown, occurs in use. In the example shown in FIG. 4, the distal surface 24 of the body portion 22 is concave so that when the insert 20 is positioned within the through hole 8, the curvature of the acetabular shell 6 is continuous across the surface of the distal surface 24 of the body portion 22.

In this embodiment, as previously described for the earlier embodiment, the through bore 28 of the insert 20 still extends through the through hole 8 of the surgical implant 6, but in doing so it narrows the diameter which receives the fixing to the d₂ diameter of the through bore 28. In this embodiment, by virtue of the tapering, from distal to proximal sides, of the through bore 28, the diameter reduces to a minimum value d₃. When the fixing, in this case a locking screw, is driven through the through bore 28 of the insert 20, the locking screw deforms the porous material that the insert 20 is formed from resulting in the head of the locking screw 22 being captured within the deformed material, locking the insert 20 in place to both the locking screw and the surgical implant 6.

As shown in FIG. 4, the through hole 8 of the surgical implant 6 is tapered. As such, the body portion 22 of the insert 20 may have a corresponding taper and a complimentary shape to the through hole 8 so that the insert 20 fills the through hole 8 once inserted.

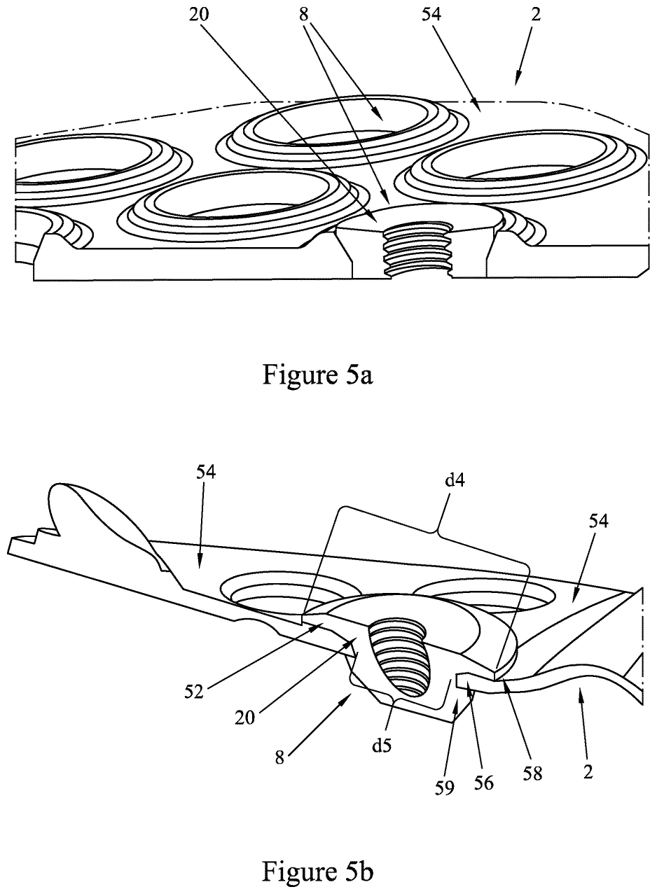

As shown in FIG. 5a, an insert 20 is shown in one of a number a through holes 8 in a plate section 54 of an acetabular cage 2. The inserts 20 can be used in a wide variety of settings to provide an adaptor and bone growth promotion.

In a related embodiment, shown in FIG. 5b, the insert 20 has a flange section 52 on the proximal side of the insert 20. The flange section 52 may be formed from a porous material for promoting bone in-growth. The insert 20 is provided in a through hole 8 in a plate section 54 of the acetabular cage 2. The diameter d₄ of the proximal flange section 52 is larger than the diameter ds of the through bore 8 in the plate section 54 at its widest. In use, this flange section 52 and particularly its distal face 56 engages with the proximal side 58 of the plate section 54. A flange section 52 is particularly useful for inserts 20 used in a cage or plate uses because the insert 20 can be positioned so that the flange section 52 sits between the surgical implant and the bone. This increases the bone contact area for promoting bone in-growth. When the flange is positioned on the upper end 12 of the body portion 4, the flange section 52 prevents the insert 2 being forced through the screw hole bore 10 of the surgical device 20. As shown, a distal flange section 59 is also provided which has a diameter greater than the diameter ds of the through bore 8. This assists with retention of the insert 20 in the acetabular cage 2.

The flange section 52 can be planar as shown in FIG. 5b, or may be concave or convex, so that it is complimentary to the surgical device into which it is intended to be inserted. For example, the flange section 52 may be concave and/or convex so that when the insert 20 is positioned within the through hole 8, the curvature of the surgical device is continuous across the surface of the flange and the upper end 12 or lower end 14 of the body portion 4.

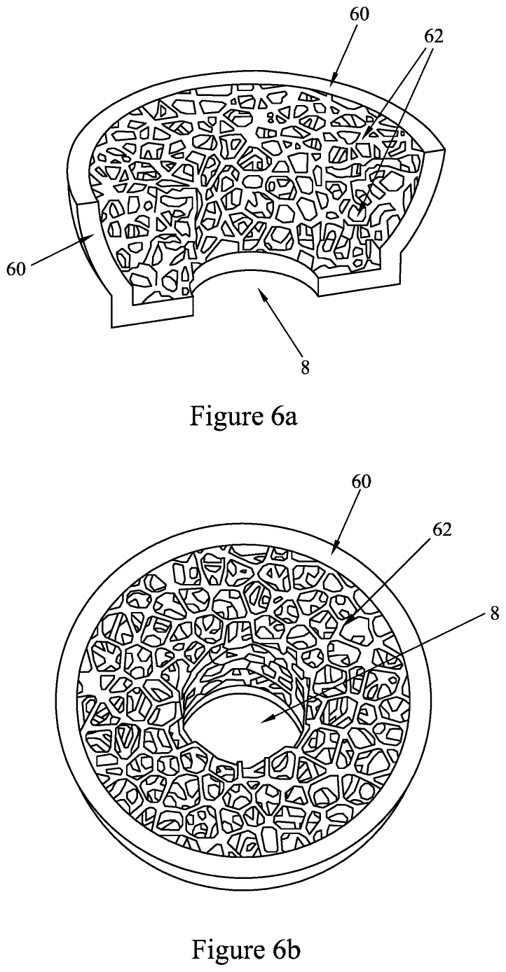

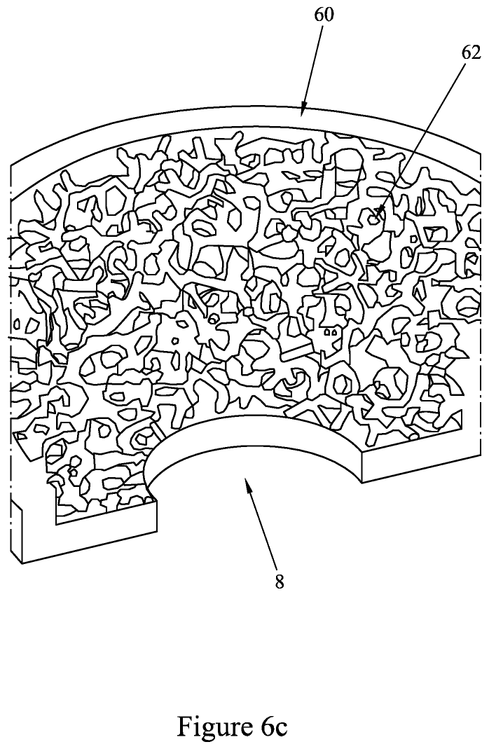

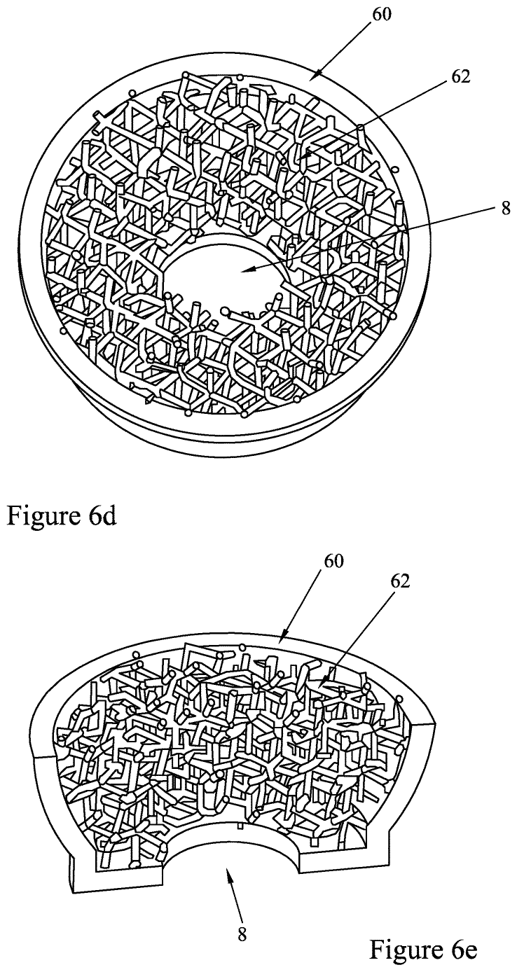

For any of the embodiments of the insert 20, the material from which it is constructed may also be deformable in surgical use. This ability to deform may apply to the entirety of the insert 20. Alternatively, in the embodiment illustrated in FIG. 6a to e, the body portion 22 of the insert is formed from different materials in different areas. For example, the outer volume 60 of the body portion 22 may be formed from a first material, for providing strength to the insert 20, whilst the inner volume 62 of the body portion 22, comprising the through bore 28, may be formed from a different material, for instance porous and/or optimised for deformation, and potentially also supporting bone ingrowth. The different material may be a non-porous material, or a porous material having different properties to the non-porous material of the outer volume 60. The material of the outer volume may be a different porous material. The different materials may be varied one another through control of the type of lattice, type of material, lattice dimensions, pore sizes or the like.

Referring to FIGS. 6a and 6b, a first construct is used, with a structural outer layer 60 and surface lattice 62 within that. The surface lattice 62 defines the proximal part of the through hole 8 and the structural outer layer 60 defines the distal part of the through hole 8. The fixing 40 would engage with both, potentially with deformation of the surface lattice 62 and/or structural layer 60.

In FIG. 6c, a second construct is used, with a structural layer 60 and a finer and so less porous 3D lattice 62 within that.

In the FIGS. 6d and 6e form, the third construct is provided with a structural outer layer 60 and a DPS Affixium™ 3D lattice 62 within.

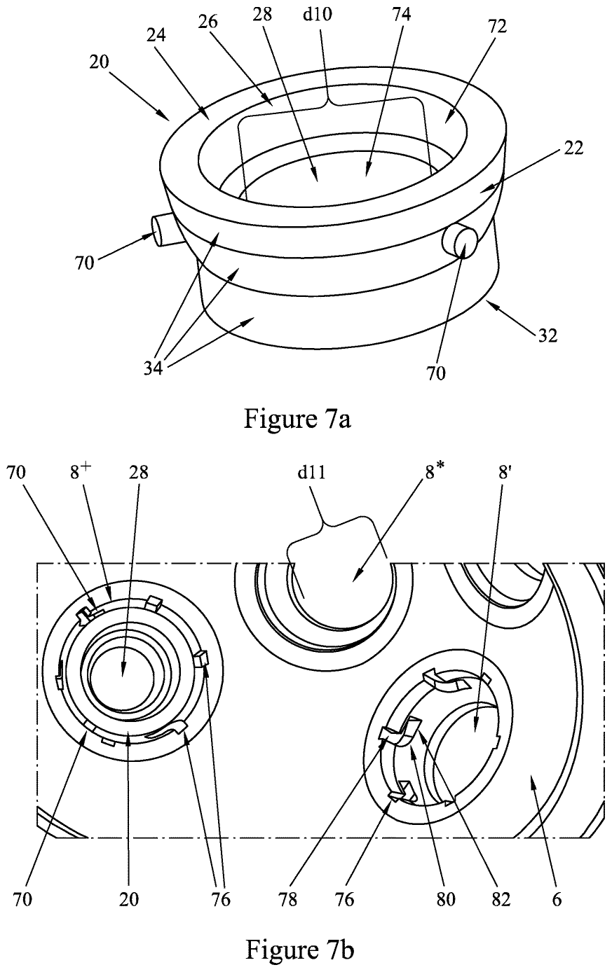

A further optional construction of the disclosure is shown in FIGS. 7a and 7b. As shown in FIG. 7a, the insert 20 comprises a body portion 22 having a distal surface 24 which is provided with a distal opening 26 located at the distal end of a through bore 28 which extends through the body portion 22 to a proximal opening 30 provided in a proximal surface 32 of the body portion 22. The through bore 28 has a diameter d₁₀ which is less than a diameter d₁₁ of the through hole 8 of the surgical implant 6, once again allowing a different size or type of fixing to be engaged with the through bore 28 and then bone.

The insert 20 in this embodiment comprises a plurality of mating features 70 provided on the outer surface 34 of the body portion 22 for locking the insert 20 into the surgical implant 2, 4, 6, for instance of the type shown in FIG. 7b. The mating features 70 can be female or male, with male forms illustrated. In the examples shown in FIGS. 7a and 7b, the mating features 70 are a bayonet-type fitting in the form of a projection from the outer surface 34. In other examples which are not shown, the mating feature can be a thread.

The insert 20 is provided with a through bore 28 which has a first section 72 and narrower section 74 closer to the proximal side. The first section 72 serves to receive the larger diameter head of the fixing, compared with the stem of the fixing in use.

The insert 20 is configured to be insertable into the through hole 8 of a variety of surgical devices, for example an acetabular shell 4, cage 2, a trauma plate, and/or a distal locking femoral stem, with the through hole 8 of the surgical implant 2, 4, 6 modified to receive the mating features 70 of the insert 20. As shown in FIG. 7b, the acetabular cup 6 in this case has a plurality of through holes 8. Some of the through holes, such as through hole 8*, are unaltered and so unsuitable to receive an insert 20 with projections 70. Through hole 8′ has been provided to a modified design and so comprises a plurality of complimentary female mating features 76, for engagement with the male mating features 70 of the insert 20. Each female mating feature 76 in this case has a first groove section 78 allowing axial movement of the male mating potion 70 relative to the insert 20, then a curved section 80 to encourage rotation of the insert 20 to start relative to the surgical implant 6 and finally a retention section 82. The retention section 82 resists any axial movement of the insert 20 and surgical implant 6 relative to one another. The retention groove section 82 is provided in a common plane, perpendicular to the axis of the through bore 8, along with the other retention groove sections 82.

An insert 20 in the retained position is shown in through hole 8⁺ with the male retention features 70 in the retention sections of the female retention parts 76.

As shown in FIG. 7a, the insert 20 provides a through bore 28 of a lesser diameter than the through hole 8′ and 8* in a surgical implant 6 thereby narrowing the diameter of the through bore 28 and hence that into which the fixing used to fasten the surgical device 2, 4, 6 and bone is inserted.

As shown in FIG. 7b, the through hole 8 of the surgical implant 6 may be tapered. As such, the proximal body portion of the insert 20 has a corresponding taper and a complimentary shape so that the insert 20 fills the through hole 8 once inserted.

In the construction shown in FIGS. 7a and 7b, the through bore 28 is unthreaded. In this construction, the thread of the fixing, such as a locking screw, may be a self-tapping screw and cut a thread into the side wall 36 of the bore to secure the fixing to the insert 20.

In other constructions which are not shown, the through bore 28 may comprise a thread formed in its side wall 36 along at least part of its length. In other constructions which are also not shown, the thread may only extend part way along the length of the side wall 36. The thread is complimentary to the thread of a fixing

The mating features 70, 76, can be positioned to allow the surgeon to alter the angle of insertion of the surgical implant 2, 4, 6 and/or of the insert 20. Alteration of the insertion angle allows the surgeon to vary the insertion angle of the through bore 28 too so that it is not aligned with the through hole 8 10 of the surgical implant 2, 4, 6. Alteration of the insertion angle allows the surgeon to insert a fixing, such as a locking or non-locking screw, at a different angle to that available in the original through hole 8, for example to access a higher quality area of bone. Varying the insertion angle may allow the insert 20 to protrude from the surgical implant 2, 4, 6, if required.

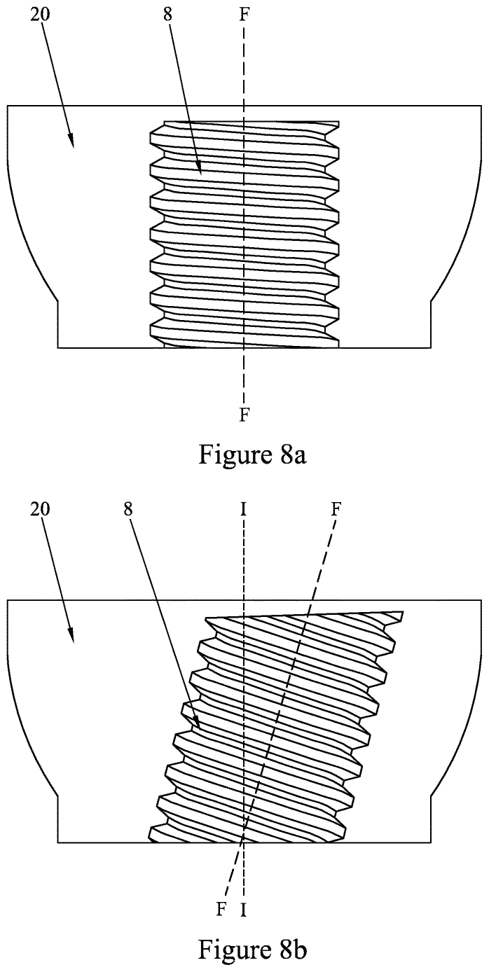

Referring to FIG. 8a and FIG. 8b, different alignment options for the through hole 8 in the insert 20 are provided. In the FIG. 8a case, the axis F-F of the through hole 8 extends distally and proximally only relative to the insert 20. Thus, the fixing 40 when inserted will follow the alignment of the axis F-F also. In the FIG. 8b case, the axis P-P of the through hole 8 extends distally and proximally, but is also offset or inclined relative to the insert 20. Thus, the fixing 40 when inserted will follow the alignment of the axis F-F and be inclined relative to the axis I-I of the insert 20.

Potentially where the insert is of a solid material and not deformable, the angle of the through hole 8 containing the corresponding locking feature, fixing 40, can be manufactured to be of zero offset or with an angular offset within a range. A variety of inserts 20 may be provided overall, with differing degrees of offset, for instance up to the level of variation available for non-locking fixing, typically 17 degrees, for example in 5 degrees steps (so 5, 10, 15 degree examples). The insert 20 can of course be rotated to different positions within the through hole 8 and so only one angle variant is needed at each offset, as the insert 8 can be rotated to allow for the required angulation in the required direction.

If the insert is deformable, there is the option to have similar pre made offset as above but with no mating feature in the through hole 8, as the locking would be through the fixing 40 biting into the material of the insert 20. In another option, the through hole 8 of the insert 20 is made of a porosity level that is high and therefore very easy to deform. Therefore, the user could just angle the fixing 40 in the through hole 8 as they desire and drive the fixing 40 through the insert 20 at the angle desired. In this case, the maximum angle would be limited by the larger aperture that receives the insert 20.

The disclosure also relates to a method of adapting a through hole 8 suitable for a first size or type of fixing, such as a non-locking screw, to be suitable for a second size of type of fixing, such as a locking screw. The method comprises the steps of first, providing a surgical implant including through holes 8, for instance for non-locking screw holes, and then providing an insert 20 according to any of the examples provided above. The insert 20 is then inserted into one of the through holes 8, for non-locking screw holes, and a second type of fixing, for instance a locking screw, is inserted into the through bore 28 of the insert 20.

There is also provided, a kit for adapting a through hole 8 suitable, for instance, for a non-locking screw into a through hole 8, for instance, suitable for a locking screw. The kit comprises at least two inserts 20 according to the insert described above and at least two different types of fixing.

Although particular embodiments of this disclosure have been described, it will be appreciated that many modifications/additions and/or substitutions may be made within the scope of the claims. While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

1. A surgical implant system, the system comprising:
one or more surgical implants, at least one of the surgical implants having a distal surface and a proximal surface, a surgical implant aperture extending between the distal surface and the proximal surface;
one or more surgical fixings; and
one or more surgical implant aperture inserts;
wherein at least one of the surgical implant aperture inserts comprises:
a body portion, the body portion providing a distal surface and a proximal surface, the body portion further providing an intermediate surface extending between the distal surface and the proximal surface;
a through bore extending between the distal surface and the proximal surface, the through bore being defined by a through bore surface;
wherein the intermediate surface includes a surgical implant aperture contacting portion;
wherein the through bore surface includes a surgical fixing contacting portion.

2. The surgical implant system of claim 1, wherein the intermediate surface defines a first cross-sectional profile in a plane, wherein the through bore surface defines a second cross-sectional profile in that plane and wherein the second profile lies within the first profile.

3. The surgical implant system of claim 1, further comprising two or more surgical fixings, including at least one fixing of a first type and at least one fixing of a second type.

4. The surgical implant system of claim 3, wherein one or more screw fixings are provided including one or more non-lockable fixings as the first type and/or one or more lockable fixings as the second type.

5. The surgical implant system of claim 3, wherein the first type of fixing is different from the second type of fixing in terms of one or more characteristics of a threaded part of the fixing

6. The surgical implant system of claim 3, wherein the first type of fixing is different from the second type of fixing in terms of one or more dimensions of the fixing, for instance a diameter of the fixing.

7. The surgical implant system of claim 1, further comprising a plurality of different surgical implant aperture inserts, the inserts being different from one another in terms of one or more or all of: the body portion; the distal surface; the proximal surface; the intermediate surface extending between the distal surface and the proximal surface; the through bore extending between the distal surface and the proximal surface; the through bore surface; the surgical implant aperture contacting portion; the surgical fixing contacting portion.

8. The surgical implant system of claim 1, further comprising a plurality of different surgical implant aperture inserts, the inserts being difference from one another in terms of one or more or all of: a material composition; a porosity; a dimension; a separation between features; a size of a feature; an extent of a feature; a shape or profile of a feature.

9. The surgical implant system of claim 1, further comprising a plurality of different surgical implant aperture inserts, the inserts being difference from one another in terms of an inclination for the bore.

10. The surgical implant system of claim 9, the insert having an insert axis, the insert axis extending distally to proximally, the through bore having a bore axis, the bore axis being inclined relative to the insert axis.

11. The surgical implant system of claim 10, wherein the inclination is between 0 degrees and 25 degrees relative to the insert axis.

12. The surgical implant system of claim 1, wherein the surgical implant is one or more of: an acetabular shell; a partial acetabular shell; an acetabular cage; a femoral implant; a trauma treating implant.

13. The surgical implant system of claim 1, wherein the surgical implant is provided with an aperture surface provided with one or more insert retention elements.

14. A surgical implant aperture insert, the insert comprising:
a body portion, the body portion providing a distal surface and a proximal surface, the body portion further providing an intermediate surface extending between the distal surface and the proximal surface;
a through bore extending between the distal surface and the proximal surface, the through bore being defined by a through bore surface;
wherein the intermediate surface includes a surgical implant aperture contacting portion;
wherein the through bore surface includes a surgical fixing contacting portion; wherein the intermediate surface defines a first cross-sectional profile in a plane, wherein the through bore surface defines a second cross-sectional profile in that plane and wherein the second profile lies within first profile.

15. The surgical implant aperture insert of claim 14, wherein at least a part of the insert is formed of a porous material, the porous material being adapted to promote bone ingrowth.

16. The surgical implant aperture insert of claim 14, wherein at least a part of the insert is formed of a deformable material, the deformable material being adapted to deform on engagement by a surgical fixing.

17. The surgical implant aperture insert of claim 14, wherein at least a part of the insert is formed of a deformable material, the deformable material having a first pre-deformation state for the through bore surface and a first post-deformation state for the through bore surface, the shape of the through bore surface being different in the first post-deformation state to the shape of the through bore surface in the first pre-deformation state.

18. The surgical implant aperture insert of claim 17, wherein the through bore surface in the first post-deformation state is adapted to engage with one or more surfaces of a surgical fixing.

19. The surgical implant aperture insert of claim 14, wherein the through bore surface includes a bone engaging surgical fixing contacting portion.

20. The surgical implant aperture insert of claim 14, wherein at least a part of the insert is formed of a deformable material, the deformable material having a second pre-deformation state for the intermediate surface and a second post-deformation state for the intermediate surface, the shape of the intermediate surface being different in the second post-deformation state to the shape of the intermediate surface in the second pre-deformation state.

21.-33. (canceled)
